(19) United States
(12) Patent Application Publication
(10) Pub. No.: US 2015/0138048 A1
(43) Pub. Date: May 21, 2015

(54) GLASSES APPARATUS USING EYESIGHT-BASED VIRTUAL IMAGE

(71) Applicant: Electronics and Telecommunications Research Institute, Daejeon (KR)

(72) Inventor: Woo Goo PARK, Daejeon (KR)

(73) Assignee: Electronics and Telecommunications Research Institute, Daejeon (KR)

(21) Appl. No.: 14/263,929

(22) Filed: Apr. 28, 2014

(30) Foreign Application Priority Data
Nov. 18, 2013 (KR) 10-2013-0139860

(51) Int. Cl. G02B 27/01; G02C 11/00; G06T 19/00; G02C 7/06; G02B 27/22; A61B 3/103; G02C 7/10

(52) U.S. Cl. CPC G02B 27/017; A61B 3/103; G02C 11/10; G02C 7/10; G02C 7/06; G02B 27/22; G06T 19/006; G02B 2027/0178
USPC 345/8; 351/206

(57) ABSTRACT

Disclosed is a glasses apparatus using an eyesight-based virtual image, including: an image photographing unit configured to adjust a focus for photographing an image based on user eyesight information; an image converter configured to convert the image photographed by the image photographing unit into a 3D image; and a display unit to which the 3D image converted by the image converter is projected.

### CROSS-REFERENCE TO RELATED APPLICATIONS

This application is based on and claims priority from Korean Patent Application No. 10-2013-0139860, filed on Nov. 18, 2013, with the Korean Intellectual Property Office, the disclosure of which is incorporated herein in its entirety by reference.

### BACKGROUND

1. Field

The present invention relates to a glasses apparatus using an eyesight-based virtual image, and more particularly to a glasses apparatus capable of providing an image matched with a current eyesight state of each user even though a user wearing the glasses apparatus is changed or eyesight information of the same user is changed by adjusting a focus of a photographing device based on eyesight information (diopter information) of the user, converting the photographed image into a 3D image, and then displaying the converted 3D image (displaying an eyesight-based virtual image).

2. Discussion of Related Art

In general, people replace several tens or several hundreds of glasses for nearsightedness and farsightedness according to personal reasons, environmental reasons, and habits in their lifetime. A person having bad eyesight wishes to more clearly view a desired letter, object, or environment by wearing glasses or lens, or a physical method, such as a Laser-Assisted In-Situ Keratomileusis (LASIK) eye surgery or a Laser-Assisted sub-epithelial Keratomileusis (LASEK) eye surgery. Most people generally wear glasses as the second best plan, than the physical method in which restoration to an original state is realistically difficult due to an aftereffect of the surgery.

Eyesight of a person is not fixed, but continuously becomes bad due to an internal factor, such as an innate/acquired disease or an external factor, such as an outside environment or a habit, for a lifetime from early years to aging years. In order to solve this, a person needs to replace several tens or several hundreds of glasses in accordance with his/her eyesight, or wear omnifocal lens in a case where nearsightedness and farsightedness coexist during a progress to farsightedness commonly exhibited in a middle-aged class. However, this requires high costs and it takes a considerable time for adapting to the omnifocal lens. Further, the most important thing is that the eyesight may be continuously changed as time goes on.

Particularly, an eyesight change is severe in childhood years or youth years due to a living environment, a reading habit, and the like, and recently, an eyesight deterioration rate of adolescents has been considerably increased according to games or craze for a smart phone. Further, in a case where a change according to eyesight deterioration of a person or a family member is neglected as it is, the eyesight may rapidly deteriorate, which is on the rise as a social problem.

Accordingly, it is very difficult and requires considerable costs to replace the large number of glasses at an accurate time in accordance with a change in an eyesight in a lifetime of a person, so that a technology for omni-glasses or universal glasses, through which a person may clearly view a world regardless of an eyesight with a pair of glasses for a lifetime of a person, is demanded.

The present invention is invented based on the aforementioned technical background, and is invented in order to provide additional technical elements which meet the aforementioned technical demands and those skilled in the art may not easily invent.

### SUMMARY

The present invention has been made in an effort to provide an omni-glasses apparatus or a universal glasses apparatus capable of displaying (providing) an image matched with a current eyesight state of each user even though a user wearing the glasses apparatus is changed or eyesight information about the same user is changed.

The technical objects to be achieved by the present invention is not limited to the aforementioned technical object, and may include various technical objects within the scope obvious to those skilled in the art from the contents to be described below.

An embodiment of the present invention provides a glasses apparatus, including: an image photographing unit configured to adjust a focus for photographing an image based on user eyesight information; an image converter configured to convert the image photographed by the image photographing unit into a 3D image; and a display unit to which the 3D image converted by the image converter is projected.

Further, the glasses apparatus according to the exemplary embodiment of the present invention may further include an eyesight test unit configured to measure eyesight of a user through a refraction test, and generate the user eyesight information.

Further, in the glasses apparatus according to the exemplary embodiment of the present invention, the eyesight test unit may include: a connection frame movably installed in a body frame; and a refraction test module formed in the connection frame, and configured to measure the eyesight of the user through the refraction test.

Further, in the glasses apparatus according to the exemplary embodiment of the present invention, the image photographing unit may measure a distance to a subject, and adjust the focus for photographing the image based on the user eyesight information and distance information.

Further, the glasses apparatus according to the exemplary embodiment of the present invention may be operated in a sunglasses mode, and an image, on which processing of polarized light filter has been performed, may be projected to the display unit in the sunglasses mode.

Further, the glasses apparatus according to the exemplary embodiment of the present invention may be operated in a zoom-in mode and a zoom-out mode, and the 3D image may be projected to the display unit in an enlarged state in the zoom-in mode and the 3D image may be projected to the display unit in a decreased state in the zoom-out mode.

Further, the glasses apparatus according to the exemplary embodiment of the present invention may further include a first sensor module configured to detect a speed, in which when a speed detected by the first sensor module belongs to a predetermined range, the zoom-in mode or the zoom-out mode is compulsorily controlled to be an inactive state.

Further, the glasses apparatus according to the exemplary embodiment of the present invention may be operated in a voice recognition mode or a connection mode with a mobile terminal device, and operated based on voice recognition information or information transmitted from the mobile terminal device.

Further, the glasses apparatus according to the exemplary embodiment of the present invention may transmit the image projected to the display unit to the mobile terminal device, or project the image transmitted from the mobile terminal device to the display unit in the connection mode with the mobile terminal device.

Further, the glasses apparatus according to the exemplary embodiment of the present invention may further include a second sensor module configured to detect a wearing state of the glasses apparatus by detecting a contact, in which the display unit may display the image only when the second sensor module detects a contact.

Another embodiment of the present invention provides a mobile terminal device, including: a communication unit configured to transceive data with a glasses apparatus for assisting eyesight; and a display unit configured to adjust a focus based on the information transceived by the communication unit and user eyesight information measured by the glasses apparatus, and then display information about a photographed image, in which the mobile terminal device is operated while being connected with a glasses apparatus assisting eyesight.

Further, in the mobile terminal device according to the exemplary embodiment of the present invention, a focus adjusting operation, a sunglasses mode activation operation, a zoom-in mode activation operation, or a zoom-out mode activation operation of the glasses apparatus may be controlled based on information transmitted by the communication unit.

Another embodiment of the present invention provides an eyesight assisting system, including: a glasses apparatus configured to adjust a focus for photographing an image based on user eyesight information, convert the photographed image into a 3D image, and displaying the converted 3D image; and a mobile terminal device configured to receive state information about the glasses apparatus, and transmit control information to the glasses apparatus.

Another embodiment of the present invention provides a method of implementing a multifocal glasses apparatus, including: (a) measuring, by a glasses apparatus, eyesight of user and generating user eyesight information; (b) adjusting, by the glasses apparatus, a focus for photographing an image by using the user eyesight information; and (c) displaying, by the glasses apparatus, an image photographed in real time.

Further, in the method of implementing the multifocal glasses apparatus, in step (c), glasses apparatus may convert the image photographed in real time into a 3D image in real time, and display the converted 3D image in real time.

Further, in the method of implementing the multifocal glasses apparatus, step (a) may include: (a-1) moving a refraction test module installed in the glasses apparatus; (a-2) measuring, by the refraction test module, the eyesight of the user through a refraction test and generating the user eyesight information; and (a-3) moving the refraction test module to an original position.

Further, in the method of implementing the multifocal glasses apparatus, step (b) may include: (b-1) measuring, by the glasses apparatus, a distance to a subject; (b-2) adjusting, by the glasses apparatus, the focus by using the user eyesight information and distance information; and (b-3) adjusting, by the glasses apparatus, the focus considering an eyesight difference between a left eye and a right eye.

Further, the method of implementing the multifocal glasses apparatus may further include (d) generating, by the glasses apparatus, an image to which a polarized light filter is applied, an image in a zoom-in state, or an image in a zoom-out state.

Another exemplary embodiment of the present invention provides a method of controlling a multifocal glasses apparatus, including: (a) transmitting user eyesight information measured by a glasses apparatus to a mobile terminal device;

(b) adjusting, by the glasses apparatus, a focus based on the user eyesight information, and then transmitting information about a photographed image to the mobile terminal device; and (c) transmitting, by the mobile terminal device, control information to the glasses apparatus.

In the meantime, the aforementioned methods may be implemented in a form of a program and then recorded in a recording medium readable by an electronic device, or provided in a form downloadable through a download server.

According to the present exemplary embodiments of the present invention, it is possible to provide an omni-glasses apparatus or a universal glasses apparatus capable of providing an image matched with a current eyesight state of each user even though a user wearing the glasses apparatus is changed or eyesight information (nearsightedness, farsightedness, and nearsightedness+farsightedness) about the same user is changed. Particularly, the present invention may adjust a focus for photographing an image in real time based on user eyesight information, and convert the photographed image into a 3D image in a state where a focus is adjusted, and provide the converted 3D image in real time, thereby providing an image matched with changed eyesight information even though the user eyesight information is changed. According to the present invention, it is possible to decrease social costs generable due to a periodical replacement of glasses in a social aspect, and it is possible to remove inconvenience in that a user needs to newly purchase glasses whenever eyesight is changed or possess a plurality of glasses according to an eyesight state in a personal aspect.

Further, according to the present invention, it is possible to improve convenience of a user through the voice recognition mode, the connection mode with a mobile terminal device, and the sunglasses mode, and especially, improve convenience or safety of a user through a connection with the sensor device. For example, the present invention may perform an operation of recognizing a glasses wearing state of a user trough various sensor devices and displaying an image only when the wearing state is recognized, and an operation of recognizing a movement speed of a user and limiting a zoom-in or zoom-out function when the movement speed exceeds a walking speed.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The above and other features and advantages of the present invention will become more apparent to those of ordinary skill in the art by describing in detail embodiments thereof with reference to the attached drawings in which:

FIG. 1 is a diagram illustrating a representative configuration of a glasses apparatus according to an exemplary embodiment of the present invention;

FIGS. 2 and 3 are views illustrating particular examples of the glasses apparatus according to the exemplary embodiment of the present invention;

FIG. 4 is a conceptual diagram illustrating an eyesight assisting system according to the exemplary embodiment of the present invention;

FIG. 5 is a diagram illustrating an example of an operation of a mobile terminal device according to the exemplary embodiment of the present invention;

FIG. 6 is a flowchart illustrating an implementation method of a multifocal glasses apparatus according to the exemplary embodiment of the present invention;

FIG. 7 is a flowchart illustrating an example of a process of generating eyesight information about a user;

FIG. 8 is a flowchart illustrating an example of a process of adjusting a photographing focus based on eyesight information about a user;

FIG. 9 is a flowchart illustrating an example of a process of a sunglasses mode, a zoom-in mode, or a zoom-out mode;

FIG. 10 is a flowchart illustrating a control method of the multifocal glasses apparatus according to the exemplary embodiment of the present invention;

FIG. 11 is a flowchart illustrating an example of a process of managing image information about the glasses apparatus by the mobile terminal device; and

FIG. 12 is a flowchart illustrating an example of a process of a voice recognition control operation.

FIG. 13 is a block diagram illustrating a computer system as an exemplary embodiment of the present invention.

### DETAILED DESCRIPTION

Hereinafter, a glasses apparatus using an eyesight-based virtual image, and a system and a method using the same according to the present invention will be described with reference to the accompanying drawings. Described exemplary embodiments are provided so that those skilled in the art may easily understand the technical spirit of the present invention, so that the present invention is not limited by the exemplary embodiments. Further, matters in the accompanying drawings are illustrated for easily describing the exemplary embodiments of the present invention, and may be different from actually implemented forms.

In the meantime, each element expressed below is an example for implementing the present invention. Accordingly, in another implementation of the present invention, another element may be used without departing from the spirit and the scope of the present invention. Further, each element may be wholly implemented only with a configuration of hardware or software, but may also be implemented by a combination of various hardware and software. Further, two or more elements may be implemented together by one hardware or software.

Further, an expression “including elements” is an open expression, and simply indicates that corresponding elements exist, and shall not be understood that additional elements are excluded.

Further, expressions, such as “first, second, . . . ” are expressions used only for the purpose of discriminating a plurality of elements, and does not limit an order between the elements or other characteristics.

Further, an expression “multifocal glasses apparatus” means a glasses apparatus capable of selectively implementing an image according to a plurality of focus states (for example, user 1—an image of focus state 1, user 2 and an image of focus state 2) in accordance with eyesight information about a user wearing the glasses apparatus.

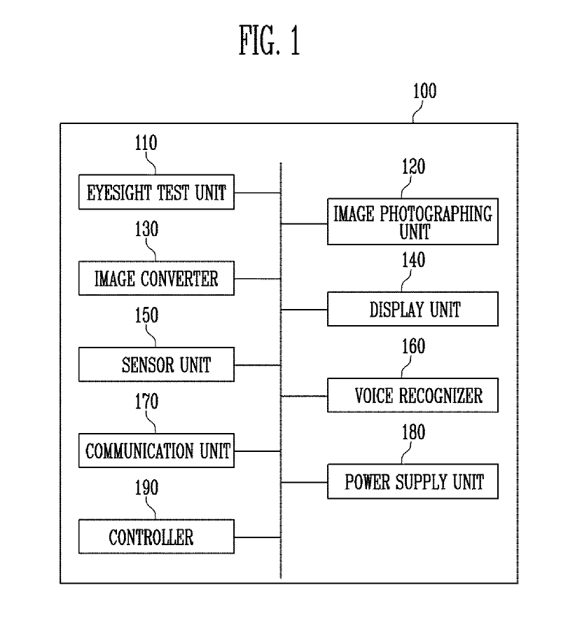

Hereinafter, a glasses apparatus 100 according to an exemplary embodiment of the present invention will be described with reference to FIGS. 1 to 3.

Referring to FIG. 1, the glasses apparatus 100 according to the exemplary embodiment of the present invention includes an eyesight test unit 110 for measuring eyesight of a user through a refraction test and generating eyesight information about the user, an image photographing unit 120 for adjusting a focus for photographing an image based on the eyesight information about the user, an image converter 130 for converting the image photographed by the image photographing unit into a 3D image; a display unit 140 to which the3D image converted by the image converter is injected, a sensor unit 150 for recognizing various state information, a voice recognizer 160 for recognizing a voice, a communication unit 170 for transceiving data; a power supply unit 180 for supplying power; and a controller 190 for controlling various elements included in the glasses apparatus.

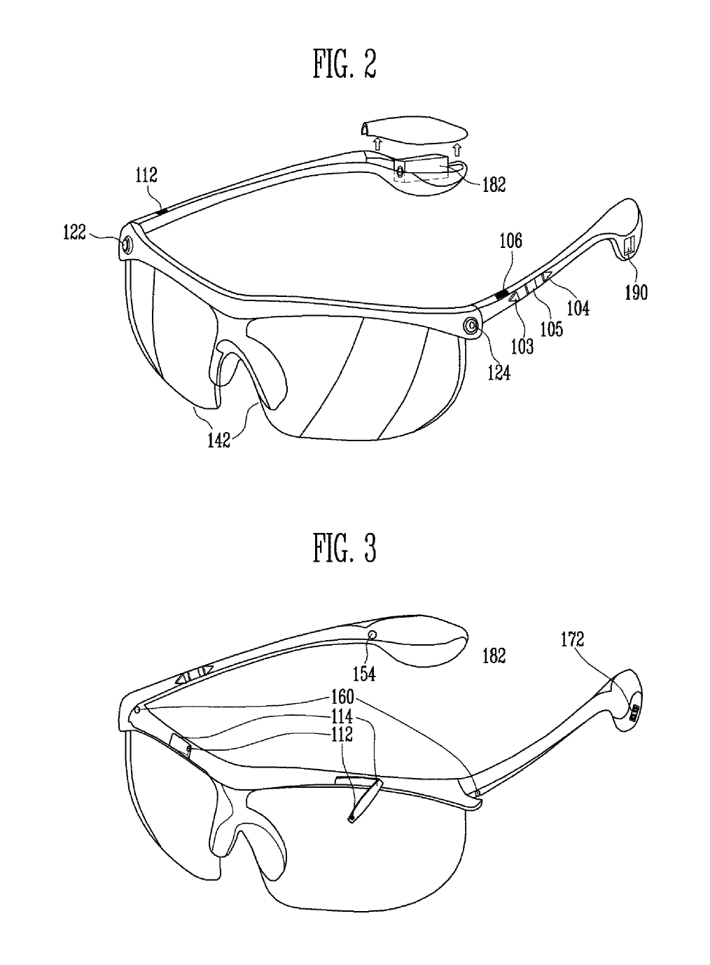

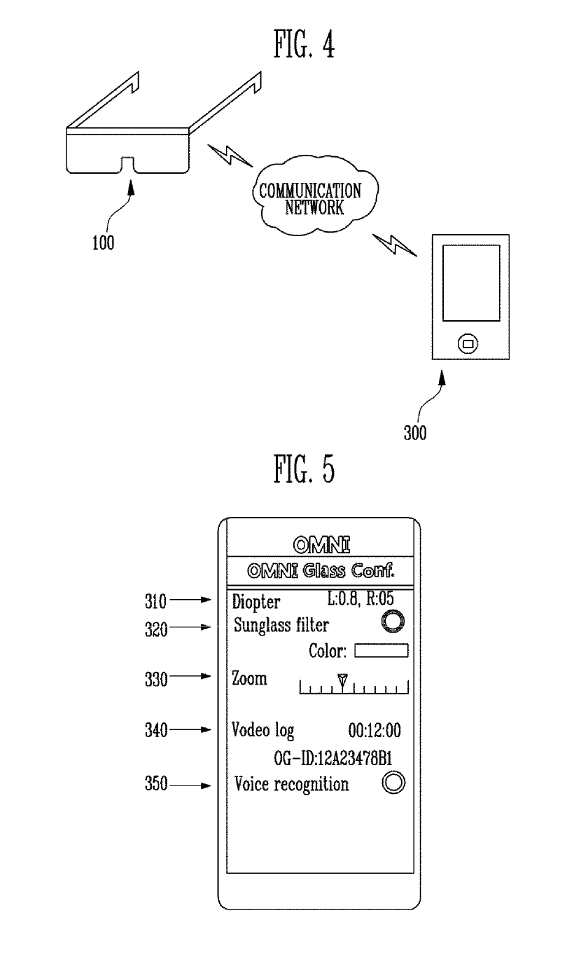

Further, the glasses apparatus 100 may further include various buttons formed in a body frame of the glasses apparatus 100 depending on an exemplary embodiment. For example, as illustrated in FIG. 2 or 3, the glasses apparatus 100 may further include an eyesight test button 112 for activating an eyesight test operation, a zoom-in button 103 for operating a zoom-in mode, a zoom-out button 104 for operating a zoom-out mode, a normal image button 105 for restoring the zoom-in or zoom-out mode to a normal state, and a sunglasses mode button 106 for operating a sunglasses mode, which are formed in the body frame of the glasses apparatus 100.

The eyesight test unit 110 is an element for measuring eyesight of the user wearing the glasses apparatus 100. The eyesight test unit 110 may measure eyesight of the user based on the refraction test, and generate eyesight information about the user based on a result of the measurement. For example, the eyesight test unit 110 may generate diopter information about eyesight of the user based on a result of the refraction test, and more preferably, may discriminate a left eye and a right eye and generate diopter information (for example, a left eye: +0.4, a right eye: −0.1).

Further, the eyesight test unit 110 may be installed in the body frame of the glasses apparatus 100, and eyesight of the user may be measured in a state where the user wears the glasses apparatus 100 through the aforementioned structure. Further, the eyesight test unit 110 may be installed in the body frame of the glasses apparatus 100 in a movable state, and may be exposed to a scope of the user's view only as necessary through the aforementioned structure. For example, the eyesight test unit 110 may include a connection frame 114 movably installed and a refraction test module 112 formed in the connection frame as illustrated in FIG. 3. Here, the connection frame 114, which is an element movably (for example, a pivot movement) installed in the body frame, is an element which is exposed to the outside (moves toward the pupil of the user) only when the eyesight test operation is performed, and is not disposed to the outside (returns to an original position in the body frame) when the eyesight test operation is not performed. Further, the refraction test module 112 is an element for measuring eyesight of the user (for example, measuring eyesight by projecting light into the eyes of the user and examining reflected light) and is an element formed in the connection frame 114 to move together with the connection frame 114. Accordingly, in the present exemplary embodiment, the eyesight test unit 110 performs an eyesight test by moving the refraction test module 112 toward the pupil of the user through the movement of the connection frame 114 only when performing the eyesight test operation, and is positioned in the body frame of the glasses apparatus 100 when not performing the eyesight test operation, thereby not disturbing the view of the user.

Further, the eyesight test unit 110 may start the eyesight test operation based on various events. For example, the eyesight test unit 110 may perform the eyesight test operation based on various event information, such as a case in which the eyesight test button 112 formed in the body frame of the glasses apparatus 100 is operated, a case in which an eyesight test command is recognized by the voice recognizer 160, a case in which a command is received from another device, such as a mobile terminal device, connected with the glasses apparatus 100, and a case in which a wearing state of the glasses apparatus 100 is detected by the sensor unit 150.

In the meantime, user eyesight information generated by the eyesight test unit 110 may be transmitted to the image photographing unit 120 or the controller 190, and may be utilized in a focus adjusting process for photographing an image.

The image photographing unit 120 is an element for performing a photographing operation based on the user eyesight information. Particularly, the image photographing unit 120 is an element for performing a photographing operation after adjusting a focus for photographing an image based on the user eyesight information. For example, when the user eyesight information is −0.5, the image photographing unit 120 performs the photographing operation after adjusting the focus in a direction of correcting a diopter of −0.5, when the user eyesight information is +0.2, the image photographing unit 120 performs the photographing operation after adjusting the focus in a direction of correcting a diopter of +0.2.

Further, the image photographing unit 120 may include a plurality of camera modules. For example, the image photographing unit 120 may include a right eye sight point camera module 122 and a left eye sight point camera module 124. In this case, the image photographing unit 120 may recognize left eyesight information and right eyesight information contained in the user eyesight information, and adjust focuses of the right eye sight point camera module 122 and the left eye sight point camera module 124 based on the recognized information.

Further, the image photographing unit 120 may also perform the focus adjusting operation considering information about a distance to a subject together. Particularly, the image photographing unit 120 may measure a distance to a subject, and then calculate an optimized focus state at corresponding eyesight and the corresponding distance considering the user eyesight information and the information about the distance. Further, the image photographing unit 120 may perform an additional focus adjusting operation based on information about a time difference between the left eye and the right eye, and may also perform the focus adjusting operation additionally considering information by the focus adjusting button (not shown) formed in the body frame of the glasses apparatus 100, information recognized by the voice recognizer 160, and information transmitted from the external device, such as the mobile terminal device 300. Accordingly, it is possible to further optimize a focus adjusting state through the focus adjusting operation considering the additional parameters.

In the meantime, it is most preferable that the image photographing unit 120 acquires the user eyesight information from the eyesight test unit 110, but the user eyesight information may be acquired through another route depending on an exemplary embodiment. For example, the image photographing unit 120 may acquire the user eyesight information based on information directly input through an input unit (not shown) which may be additionally included in the glasses apparatus 100, the information recognized by the voice recognizer 160, or the information transmitted from the external device, such as the mobile terminal device.

The image converter 130 is an element for converting an image photographed by the image photographing unit 120 into a 3D image. The image converter 130 may convert a left eye photographing image and a right eye photographing image photographed by the image photographing unit 120 to a single 3D image, and in this case, information about a distance to a subject generated by the image photographing unit 120 may be utilized in a converting process. Further, the image converter 130 may render a virtual image to be projected to the display unit 140 based on the converted 3D image data, and in this case, the image converting unit 130 may render the virtual image considering a form of the display unit 140.

In the meantime, the image converter 130 may include various image processing modules.

The display unit 140 is an element visually displaying the image converted by the image converter 130. The image displayed by the display unit 140 is the image photographed after the focus is adjusted based on the user eyesight information and then 3D converted, so that the user of the glasses apparatus 100 may feel an effect as if wearing the existing glasses with power through the image displayed by the display unit 140.

In the meantime, the display unit 140 may be implemented by various display devices, such as an LCD and an LED, but may be preferably implemented by a transparent display device. For example, the display unit 140 may be implemented by a transparent Organic Light Emitting Diode (OLED) device.

The sensor unit 150 is an element for recognizing various state information of the glasses apparatus 100. The sensor unit 150 may include a first sensor module for recognizing speed information about the glasses apparatus 100, and a second sensor module for recognizing a wearing state of the glasses apparatus 100, and may include various sensor modules in addition to the aforementioned sensor modules.

First, the first sensor module is an element for recognizing speed information about the glasses apparatus 100. The first sensor module may include various sensors, such as a 3-axis acceleration sensor, a gyro sensor, and a Global Positioning System (GPS) sensor, and recognize the speed information about the glasses apparatus 100 (the body itself of the glasses apparatus 100) through the sensors. Particularly, the sensor unit 150 may generate the speed information about a movement of the user wearing the glasses apparatus 100 by using the first sensor module.

Further, the second sensor module is an element for recognizing a wearing state of the glasses apparatus 100. The second sensor module may include various contact detecting sensors, such as a capacitive touch sensor or a resistive touch sensor, and may recognize the wearing state of the glasses apparatus 100 by using the contact detecting sensor. Referring to FIG. 3, an example of the second sensor module is illustrated. In the exemplary embodiment of FIG. 3, the second sensor module detects a contact (a contact around a temple) generated in a state where the glasses apparatus 100 is worn, and generates the information about the wearing state of the glasses apparatus 100 through the detected contact.

The information generated by the sensor unit 150 may be utilized in various control processes. For example, the speed information generated by the first sensor module may be utilized in a process of controlling a zoom-in mode or a zoom-out mode to be described below, and the information generated by the second sensor module may be utilized in a process of controlling general on/off of the glasses apparatus 100 (for example, controlling the glasses apparatus 100 to be on only in a state where the glasses apparatus 100 is worn).

The voice recognizer 160 is an element for performing a voice recognition operation. Particularly, the voice recognizer 160 is an element for recognizing a voice of the user and then performing an operation of analyzing the recognized voice. Referring to FIG. 3, an example of the voice recognizer 160 is illustrated.

The voice recognizer 160 may include a microphone module for receiving a voice and an analysis module for interpreting the input voice, and may extract various information contained in the voice of the user and reflect the extracted information to the operation of the glasses apparatus 100 through the element. Further, the microphone module and the analysis module may be implemented in various formed within the scope apparent to those skilled in the art.

The communication unit 170 is an element for transmitting or receiving various data to or from the external device, such as the mobile terminal device. The communication unit 170 may transmit various information generated by a guiding device to the external device, or receive control information capable of controlling an operation of the guide device to the external device.

In the meantime, the communication unit 170 may include various wired communication modules or wireless communication modules. For example, the communication unit 170 may include a USB port 172 as illustrated in FIG. 3, or a wireless communication module for processing wireless data, such as Wi-Fi, Bluetooth, and Near Field Communication (NFC), and may include various wired communication modules or wireless communication modules in addition to the example.

The power supply unit 180 is an element for supplying power for operating the glasses apparatus 100. Particularly, the power supply unit 180 supplies electricity for operating various elements included in the glasses apparatus 100.

The power supply unit 180 may be implemented in a form including various types of power devices, and may be implemented in a form including a small battery.

The control unit 190 is an element for controlling various elements of the glasses apparatus 100 including the eyesight test unit 110, the image photographing unit 120, the image converting unit 130, the display unit 140, the sensor unit 150, the voice recognizer 160, the communication unit 170, the power supply unit 180, and the various buttons.

The controller 190 may include at least one calculation device, and here, the calculation device may include a common Central Processing Unit (CPU), a programmable device element (Complex Programmable Logic Device (CPLD), Field Programmable Gate Array (FPGA)) implemented to be suitable to a specific purpose, a semiconductor calculation device on demand (Application Specific Integrated Circuit (ASIC)), or a micro controller chip.

The aforementioned glasses apparatus 100 according to the exemplary embodiment of the present invention may adjust a focus for photographing an image based on the user eyesight information in real time, and convert the image photographed in a state where the focus is adjusted to a 3D image, and then display the converted 3D image in real time. Accordingly, even though the user eyesight information (nearsightedness, farsightedness, and nearsightedness+farsightedness) is changed, the glasses apparatus 100 may provide an image matched with the changed eyesight information.

Further, the aforementioned glasses apparatus 100 may be operated in a zoom-in mode or a zoom-out mode. Particularly, the glasses apparatus 100 may be operated in the zoom-in mode or the zoom-out mode based on information input through the zoom-in button 103 and the zoom-out button 104 formed in the body frame of the glasses apparatus 100, the information recognized by the voice recognizer 160, the information transmitted from the external device, such as the mobile terminal device.

Here, the zoom-in mode is a mode displaying an enlarged image of the photographed image, and the zoom-out mode is a mode displaying a decreased image of the photographed image. The zoom-in mode or the zoom-out mode may be implemented through an operation of enlarging or decreasing the 3D image converted by the image converter 130 and then projecting the enlarged or decreased 3D image to the display unit 140.

In the meantime, when the zoom-in mode or the zoom-out mode is activated in a state where the user runs or drives a transporting device, such as a vehicle, a safety problem may be generated to the user. Further, when the user runs or drives a transporting device, such as a vehicle in a state where the zoom-in mode or the zoom-out mode is activated, a safety problem may also be generated to the user. Accordingly, a technique for preventing the problem is required. The glasses apparatus 100 may solve the safety problem through a control operation based on the generation generated by the sensor unit 150. For example, when the speed information about the user recognized through the first sensor module exceeds a predetermined walking speed (for example, 3 km/h), even though the control information activating the zoom-in mode or the zoom-out mode is input (or transmitted), the glasses apparatus 100 compulsorily suppresses the activation of the zoom-in mode or the zoom-out mode, thereby securing safety of the user. Further, when the speed information about the user recognized through the first sensor module exceeds a predetermined walking speed in a state where the zoom-in mode or the zoom-out mode is activated, the glasses apparatus 100 compulsorily closes the zoom-in mode or the zoom-out mode and controls so that an image in a general state is displayed, thereby securing safety of the user.

Further, the glasses apparatus 100 may be operated in a sunglasses mode. Particularly, the glasses apparatus 100 may also provide the user with an effect as if the user wears sunglasses by performing processing of a polarized light filter on the image projected to the display unit 140. The glasses apparatus 100 may be operated in the sunglasses mode based on the sunglasses mode button 106, the information recognized by the voice recognizer 160, the information transmitted from the external device, such as the mobile terminal device.

In the meantime, the glasses apparatus 100 may perform a protection operation of compulsorily activating the sunglasses mode based on luminance information about the image. For example, when luminance of the image to be projected to the display unit 140 exceeds a predetermined value, the glasses apparatus 100 may compulsorily activate the sunglasses mode in view of protection of a sense of sight.

Further, the glasses apparatus 100 may be operated with the mobile terminal device in a connected state. For example, the glasses apparatus 100 may transmit the information generated by the eyesight test unit 110, the image photographing unit 120, the image converter 130, the sensor unit 150, and the voice recognizer 160 to the mobile terminal device, or receive various information (control information and the like) related to the operation of the glasses apparatus 100 from the mobile terminal device.

Further, the glasses apparatus 100 and the mobile terminal device may perform a connecting operation of exchanging an image therebetween. For example, the glasses apparatus 100 may transmit the image projected to the display unit 140 to the mobile terminal device, and make the image projected to the display unit 140 be displayed in the mobile terminal device. Further, the mobile terminal device may transmit image data photographed by a photographing unit thereof or image data stored in a memory thereof to the glasses apparatus 100, and make the image data be displayed through the display unit 140 of the glasses apparatus 100.

Hereinafter, an eyesight assisting system according to the exemplary embodiment of the present invention will be described with reference to FIGS. 4 and 5.

Referring to FIG. 4, the eyesight assisting system according to the exemplary embodiment of the present invention may include the glasses apparatus 100 for adjusting a focus for photographing an image based on the user eyesight information, converting the photographed image into a 3D image, and then displaying the converted 3D image, and the mobile terminal device 300 for receiving state information about the glasses apparatus 100 and transmitting control information about the glasses apparatus 100.

Here, the glasses apparatus 100 has been described in detail, so that hereinafter, an operation of the mobile terminal device 300 will be mainly described.

The mobile terminal device 300 is a device capable of transceiving various information with the glasses apparatus 100, and being operated in a state of being connected state with the glasses apparatus 100. The mobile terminal device may be implemented by a smart phone, a tablet PC, a PDA, and a notebook computer in which a program or an application is installed, or in a form of a dedicated terminal device.

Further, the mobile terminal device 300 may includes a communication unit for transceiving data with the glasses apparatus 100 for assisting eyesight, and a display unit for displaying user eyesight information measured by the glasses apparatus 100 and information about the image photographed after adjusting a focus based on the user eyesight information by the glasses apparatus 100 based on the information transceived by the communication unit, and in addition to the aforementioned elements, the mobile terminal device 300 may various elements, such as an input unit, a storage unit, and a controller.

The mobile terminal device 300 may receive various state information about the glasses apparatus 100 and display the received state information. Particularly, the mobile terminal device 300 may receive and display various state information, such as the user eyesight information, the information about the sunglasses mode, and the information about the zoom-in mode and the zoom-out mode serving as bases of the glasses apparatus 100, the state information about the image displayed on the display unit of the glasses apparatus 100, and the information about the voice recognition mode. A particular example of displaying various state information of the glasses apparatus 100 by the mobile terminal device 300 will be described with reference to FIG. 5. As can be seen in FIG. 5, the mobile terminal device 300 may display various state information, such as user eyesight information 310, information 320 about the sunglasses mode, and information 330 about the zoom-in mode and the zoom-out mode, state information 340 about the image displayed on the display unit of the glasses apparatus 100, and information about the voice recognition mode 350.

Further, the mobile terminal device 300 may receive and manage image information generated by the glasses apparatus 100. For example, the mobile terminal device 300 may receive the image data projected to the display unit of the glasses apparatus 100 and display the received image data in the display unit of the mobile terminal device 300 or store the received image data in the storage unit of the mobile terminal device 300.

Further, the mobile terminal device 300 may transmit the control information for controlling the operation of the glasses apparatus 100 to the glasses apparatus 100, and make the operation of the glasses apparatus 100 be controlled by the control information. For example, the mobile terminal device 300 may transmit the control information for controlling the focus adjusting operation, the activation operation of the sunglasses mode, the activation operation of the zoom-in mode, or the activation operation of the zoom-out mode of the glasses apparatus 100 to the glasses apparatus 100, and make each operation of the glasses apparatus 100 be controlled by the control information.

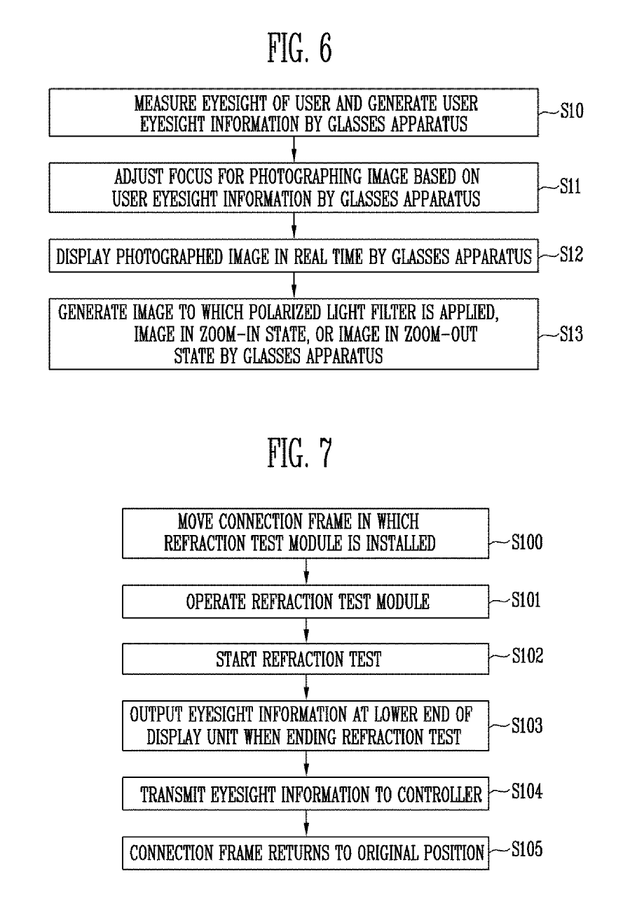

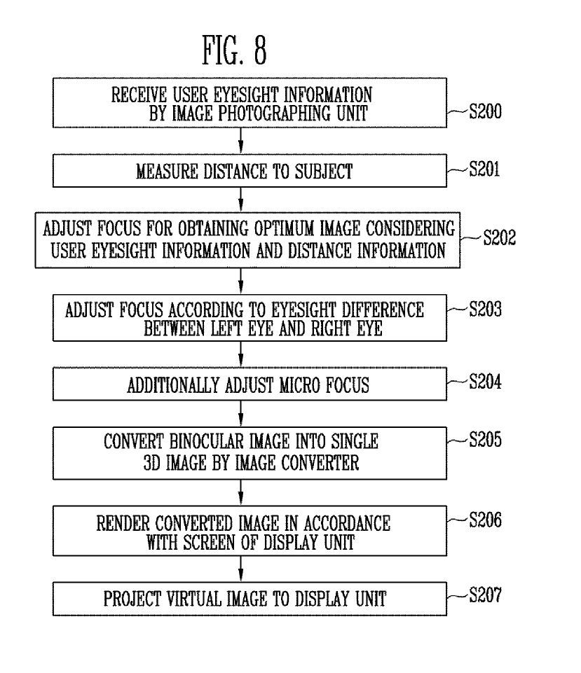

Hereinafter, a method of implementing the multifocal glasses apparatus according to an exemplary embodiment of the present invention will be described with reference to FIG. 6.

Referring to FIG. 6, the method of implementing the multifocal glasses apparatus according to the exemplary embodiment of the present invention may include step S10 of measuring, by the glasses apparatus, eyesight of a user and generating user eyesight information.

Further, after step S10, the method may further include step S11 of adjusting, by the glasses apparatus, a focus for photographing an image based on the user eyesight information.

Further, after step S11, the method may further include step S12 of displaying, by the glasses apparatus, an image photographed in real time. Particularly, the method may further include a step of converting an image photographed in real time, converting the photographed image into a 3D image, and displaying the converted 3D image in real time by the glasses apparatus.

Further, after step S12, the method may further include step S13 of generating an image to which a light polarization filter is applied, generating an image in a zoom-in state, or generating an image in a zoom-out state by the glasses apparatus.

In the meantime, step S10 may include step S10-1 of moving the refraction examination module installed in the glasses apparatus as a detailed process. Further, step S10 may further include step S10-2 of measuring, by the refraction test module, eyesight of the user through a refraction test and generating the user eyesight information after step S10-1. Further, step S10 may further include step S10-3 of moving the refraction test module to an original position after step S10-2.

Further, step S11 may include step S11-1 of measuring, by the glasses apparatus, a distance to a subject as a detailed process. Further, step S11 may further include step S11-2 of adjusting, by the glasses apparatus, the focus by using the user eyesight information and distance information after step S11-1. Further, step S11 may further include step S11-3 of adjusting, by the glasses apparatus, the focus considering an eyesight difference between a left eye and a right eye.

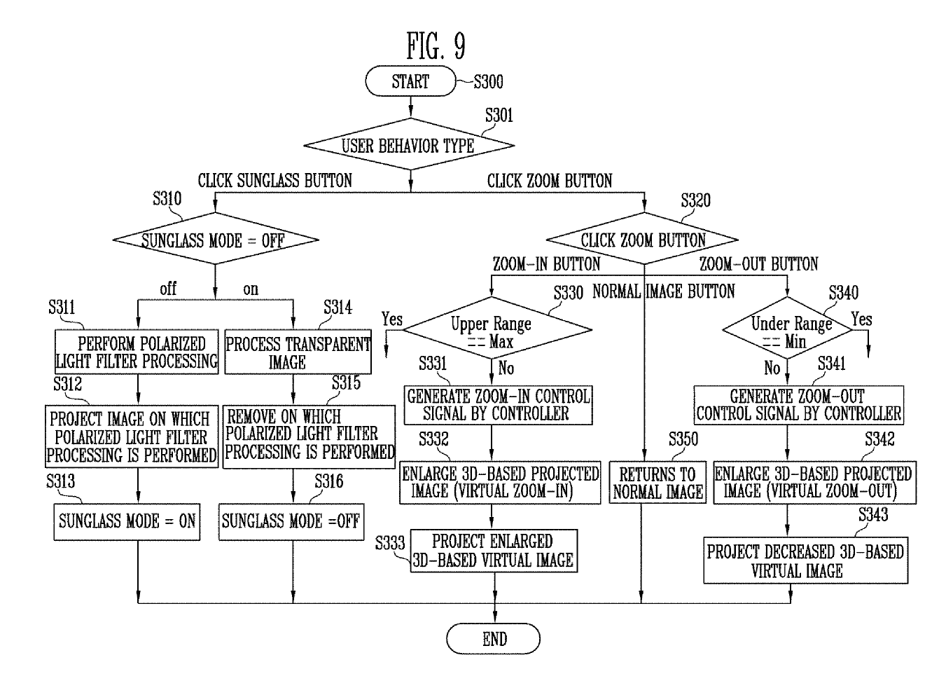

Hereinafter, a detailed example of step S10 will be described with reference to FIG. 7.

Referring to FIG. 7, a connection frame installed in the refraction test module among the elements of the glasses apparatus moves, and the refraction test module moves through the movement of the connection frame (S100). In this case, the refraction test module may move to a position of the pupil of the user wearing the glasses apparatus.

After the refraction test module moves to the position of the pupil of the user, the refraction test module is operated (S101), and a refraction test is started (S102). Particularly, the refraction test module measures the eyesight of the user through the refraction test and generates the user eyesight information.

After the refraction test module generates the user eyesight information, the refraction test is ended, and the generated user eyesight information is output at a lower end of the display unit of the glasses apparatus (S103). Further, the user eyesight information is transmitted to the controller (S104), and the refraction test module returns to an original position through the movement of the connection frame (S105).

Hereinafter, detailed examples of step S11 and step S12 will be described with reference to FIG. 8.

Referring to FIG. 8, the image photographing unit among the elements of the glasses apparatus receives the user eyesight information (S200). After the user eyesight information is received, the image photographing unit measures a distance to the subject (S201), and performs the focus adjusting operation for obtaining an optimum image considering the user eyesight information and the distance information (S202). Further, the image photographing unit performs the focus adjusting operation considering information about the eyesight difference between the left eye and the right eye (S203), and performs additional micro focus adjustment based on the voice recognition and the like (S204).

After the focus is adjusted, the image photographing unit photographs a subject by using the adjusted focus, and the image converter converts the image photographed by the image photographing unit. Particularly, the image converter converts a binocular image to a single 3D image (S205). The image converted by the image converter is rendered in accordance with a screen of the display unit of the glasses apparatus (S206), and is projected to the display unit (S207).

Hereinafter, a detailed example of step S13 will be described with reference to FIG. 9.

Referring to FIG. 9, the operation of the glasses apparatus is started (S300), and a command of the user is recognized. Here, the recognition of the command of the user may be implemented by various manners, such as a button, voice recognition, and reception of data through a communication network, but in the example of FIG. 9, the recognition of the command by the button will be representatively described.

First, a behavior type of the user is analyzed (S301), and when a sunglasses button is activated (S301—Yes) as a result of the analysis, the glasses apparatus confirms whether the sunglasses mode is in an off-state (S310). 1) When the sunglasses mode is in the off state, the glasses apparatus performs an operation of filtering polarized light on the image, projects the image on which processing of the polarized light filter is performed, and switches the sunglasses mode to an on-state. When the sunglasses mode is not in the off state (S301—No), the glasses apparatus performs a transparent image processing operation, removes the image on which the processing of the polarized light filter has been performed, and switches the sunglasses mode to the off state.

Next, when the zoom-in button, the zoom-out button, or the normal image button is activated (S320) as the result of the analysis (S301) of the behavior state of the user, the glasses apparatus performs an operation related to the zoom. 1) When the zoom-in button is activated, the glasses apparatus confirms whether the zoom-in operation is performed to a limit state (S330). When the zoom-in operation is not performed to the limit state as a result of the confirmation, the controller of the glasses apparatus generates a zoom-in control signal (S331), and thus, the 3D-gased projected image is enlarged (S332), and the enlarged 3D-gased image is projected (S333). In the meantime, when the zoom-in operation is performed to the limit state as the result of the confirmation, the glasses apparatus ends the operation. 2) When the zoom-out button is activated, the glasses apparatus confirms whether the zoom-out operation is performed to a limit state (S340). When the zoom-out operation is not performed to the limit state as a result of the confirmation, the controller of the glasses apparatus generates a zoom-out control signal (S341), and thus, the 1D-gased projected image is decreased (S342), and the enlarged 3D-gased image is projected (S343). In the meantime, when the zoom-out operation is performed to the limit state as the result of the confirmation, the glasses apparatus ends the operation.

In the meantime, FIGS. 7 to 9 are the detailed examples suggested for helping the understanding, and the method of implementing the multifocal glasses apparatus is not limited by the examples.

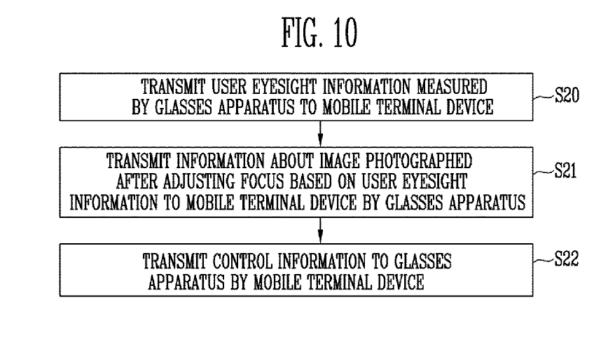

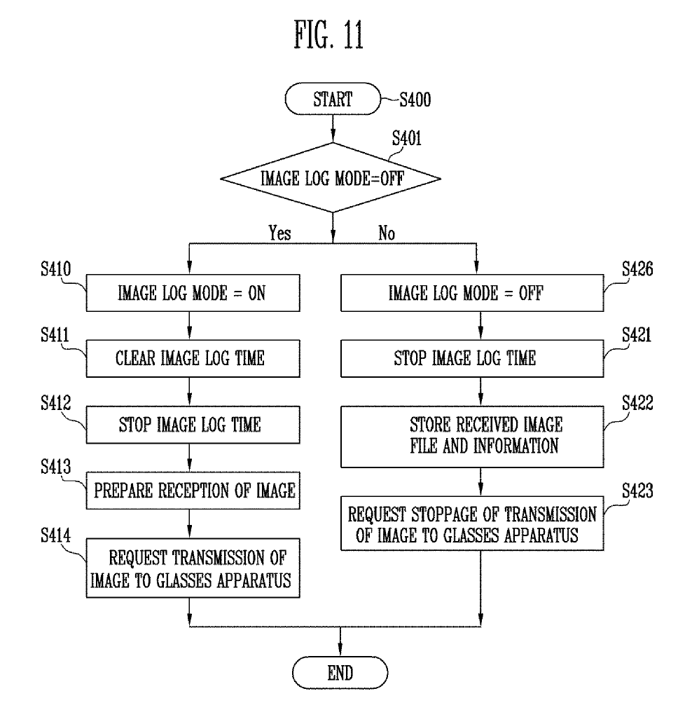

Hereinafter, a method of controlling the multifocal glasses apparatus according to an exemplary embodiment of the present invention will be described with reference to FIG. 10.

Referring to FIG. 10, the method of controlling the multifocal glasses apparatus according to the exemplary embodiment of the present invention may include step S20 of transmitting user eyesight information measured by the glasses apparatus to the mobile terminal device.

Further, after step S20, the method may further include step S21 of transmitting information about an image photographed after adjusting a focus based on the user eyesight information to the mobile terminal device.

Further, after step S21, the method may further include step S22 of transmitting, by the mobile terminal device, control information to the glasses apparatus.

Hereinafter, a detailed example of step S21 will be described with reference to FIG. 11.

Referring to FIG. 11, the mobile terminal device starts a connecting operation (S400), and confirms whether an image log mode is in an off-state (S401). 1) When the image log mode is in the off-state as a result of the confirmation (S401—Yes), the mobile terminal device switches the image log mode to an on-state (S410), clears an image log time (S411), and starts the image log time (S412). Further, reception of the image is prepared (S413), and then transmission of the image is requested to the glasses apparatus (S414). 2) When the image log mode is not in the off-state as the result of the confirmation (S401—No), the mobile terminal device switches the image log mode to an off-state (S420), stops the image log time (S421), and stores a received image file and information (S422). Further, the mobile terminal device makes a request for stoppage of the transmission of the image to the glasses apparatus (S423).

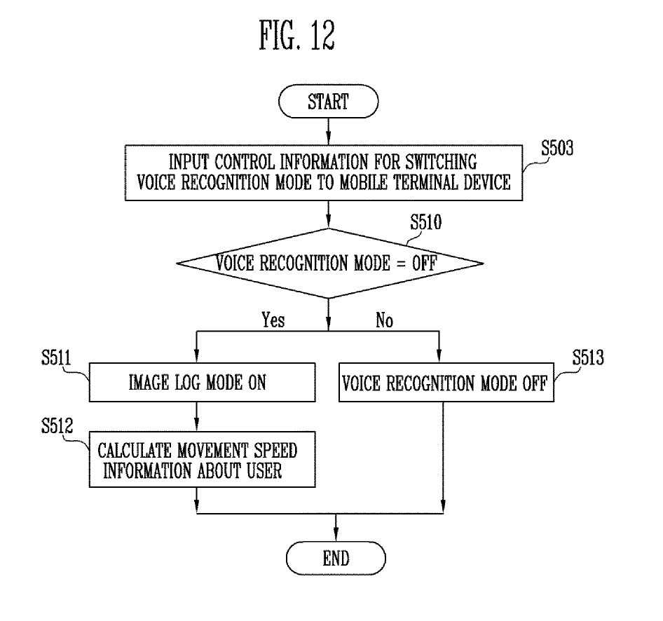

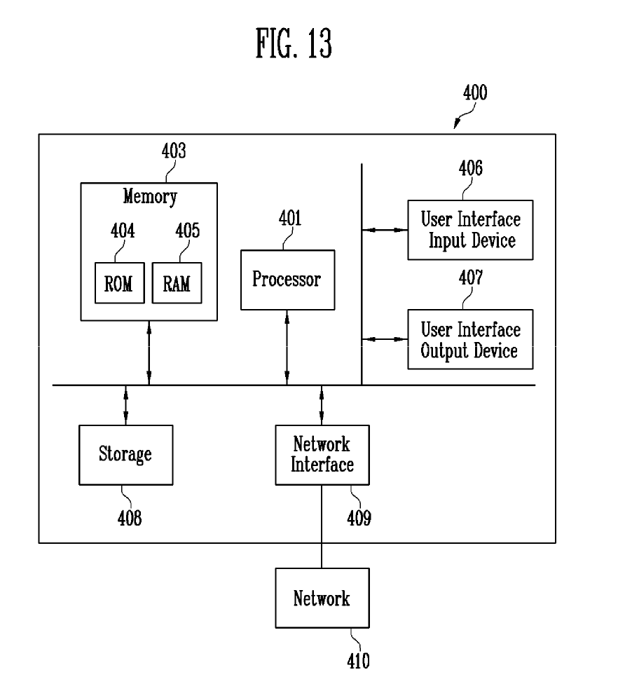

Hereinafter, a detailed example of step S21 will be described with reference to FIG. 12.

Referring to FIG. 12, control information for setting the switching of the voice recognition mode of the glasses apparatus may be input to the mobile terminal device (S503), and the input control information may be transmitted to the glasses apparatus.

Here, 1) when the voice recognition mode of the glasses apparatus is in an off state (S510—Yes), the voice recognition mode of the glasses apparatus is set to be switched to an on-state by the control information transmitted from the mobile terminal device. Further, movement speed information about the user is calculated. (For reference, the movement speed information may be calculated by the glasses apparatus or by the mobile terminal device, and then transmitted to the glasses apparatus.) Further, 2) when the voice recognition mode of the glasses apparatus is not in the off state (S510—No), the voice recognition mode of the glasses apparatus is set to be switched to an off-state by the control information transmitted from the mobile terminal device.

In the meantime, FIGS. 11 and 12 are the detailed examples suggested for helping the understanding, and the method of controlling the multifocal glasses apparatus is not limited by the examples.

In the meantime, the method of implementing the multifocal glasses apparatus or the method of controlling the multifocal glasses apparatus according to the exemplary embodiment of the present invention may be implemented in a form of a program, and may be recorded in a recording medium readable by an electronic device or be provided in a form downloadable by a download server after being implemented in the form of the program.

Further, the method of implementing the multifocal glasses apparatus or the method of controlling the multifocal glasses apparatus according to the exemplary embodiment of the present invention may have a different category, but include substantially the same technical characteristic as that of the glasses apparatus, the eyesight assisting system or the mobile terminal device according to the exemplary embodiment of the present invention.

Accordingly, although not described in detail in order to prevent overlapping descriptions, the aforementioned characteristics related to the glasses apparatus, the eyesight assisting system or the mobile terminal device may be inferred and applied to the method of implementing the multifocal glasses apparatus or the method of controlling the multifocal glasses apparatus according to the exemplary embodiment of the present invention as a matter of course. Further, the aforementioned characteristic related to the method of implementing the multifocal glasses apparatus or the method of controlling the multifocal glasses apparatus may be inferred and applied to the glasses apparatus, the eyesight assisting system or the mobile terminal device as a matter of course.

An embodiment of the present invention may be implemented in a computer system, e.g., as a computer readable medium. As shown in FIG. 13, a computer system 400 may include one or more of a processor 401, a memory 403, a user input device 406, a user output device 407, and a storage 408, each of which communicates through a bus 402. The computer system 400 may also include a network interface 409 that is coupled to a network 410. The processor 401 may be a central processing unit (CPU) or a semiconductor device that executes processing instructions stored in the memory 403 and/or the storage 408. The memory 403 and the storage 408 may include various forms of volatile or non-volatile storage media. For example, the memory may include a read-only memory (ROM) 404 and a random access memory (RAM) 405.

Accordingly, an embodiment of the invention may be implemented as a computer implemented method or as a non-transitory computer readable medium with computer executable instructions stored thereon. In an embodiment, when executed by the processor, the computer readable instructions may perform a method according to at least one aspect of the invention.

As described above, the embodiment has been disclosed in the drawings and the specification. The specific terms used herein are for purposes of illustration, and do not limit the scope of the present invention defined in the claims. Accordingly, those skilled in the art will appreciate that various modifications and another equivalent example may be made without departing from the scope and spirit of the present disclosure. Therefore, the sole technical protection scope of the present invention will be defined by the technical spirit of the accompanying claims.

What is claimed is:

1. A glasses apparatus for assisting eyesight, comprising:
an image photographing unit configured to adjust a focus for photographing an image based on user eyesight information;
an image converter configured to convert the image photographed by the image photographing unit into a 3D image; and
a display unit to which the 3D image converted by the image converter is projected.

2. The glasses apparatus of claim 1, further comprising:
an eyesight test unit configured to measure eyesight of a user through a refraction test, and generate the user eyesight information.

3. The glasses apparatus of claim 2, wherein the eyesight test unit includes:
a connection frame movably installed in a body frame; and
a refraction test module formed in the connection frame, and configured to measure the eyesight of the user through the refraction test.

4. The glasses apparatus of claim 1, wherein the image photographing unit measures a distance to a subject, and adjusts the focus for photographing the image based on the user eyesight information and distance information.

5. The glasses apparatus of claim 1, wherein the glasses apparatus is operated in a sunglasses mode, and an image, on which processing of polarized light filter has been performed, is projected to the display unit in the sunglasses mode.

6. The glasses apparatus of claim 1, wherein the glasses apparatus is operated in a zoom-in mode and a zoom-out mode, and
the 3D image is projected to the display unit in an enlarged state in the zoom-in mode and
the 3D image is projected to the display unit in a decreased state in the zoom-out mode.

7. The glasses apparatus of claim 6, further comprising:
a first sensor module configured to detect a speed,
wherein when a speed detected by the first sensor module belongs to a predetermined range, the zoom-in mode or the zoom-out mode is compulsorily controlled to be an inactive state.

8. The glasses apparatus of claim 1, wherein the glasses apparatus is operated in a voice recognition mode or a connection mode with a mobile terminal device, and
is operated based on voice recognition information or information transmitted from the mobile terminal device.

9. The glasses apparatus of claim 8, wherein in the connection mode with the mobile terminal device, the glasses apparatus transmits the image projected to the display unit to the mobile terminal device, or projects the image transmitted from the mobile terminal device to the display unit.

10. The glasses apparatus of claim 1, further comprising:
a second sensor module configured to detect a wearing state of the glasses apparatus by detecting a contact,
wherein the display unit displays the image only when the second sensor module detects a contact.

11. A mobile terminal device, comprising:
a communication unit configured to transceive data with a glasses apparatus for assisting eyesight; and
a display unit configured to adjust a focus based on the information transceived by the communication unit and user eyesight information measured by the glasses apparatus, and then display information about a photographed image,
wherein the mobile terminal device is operated while being connected with a glasses apparatus assisting eyesight.

12. The mobile terminal device of claim 11, wherein a focus adjusting operation, a sunglasses mode activation operation, a zoom-in mode activation operation, or a zoom-out mode activation operation of the glasses apparatus is controlled based on information transmitted by the communication unit.

13. A method of implementing a multifocal glasses apparatus, comprising:
(a) measuring, by a glasses apparatus, eyesight of user and generating user eyesight information;
(b) adjusting, by the glasses apparatus, a focus for photographing an image by using the user eyesight information; and
(c) displaying, by the glasses apparatus, an image photographed in real time.

14. The method of claim 13, wherein in step (c), the glasses apparatus converts the image photographed in real time into a 3D image in real time, and displays the converted 3D image in real time.

15. The method of claim 13, wherein step (a) includes:
(a-1) moving a refraction test module installed in the glasses apparatus;
(a-2) measuring, by the refraction test module, the eyesight of the user through a refraction test and generating the user eyesight information; and
(a-3) moving the refraction test module to an original position.

16. The method of claim 13, wherein step b) includes:
(b-1) measuring, by the glasses apparatus, a distance to a subject;
(b-2) adjusting, by the glasses apparatus, the focus by using the user eyesight information and distance information; and
(b-3) adjusting, by the glasses apparatus, the focus considering an eyesight difference between a left eye and a right eye.

17. The method of claim 13, further comprising:
(d) generating, by the glasses apparatus, an image to which a polarized light filter is applied, an image in a zoom-in state, or an image in a zoom-out state.
